(19) United States
(12) Patent Application Publication
(10) Pub. No.: US 2012/0276199 A1
(43) Pub. Date: Nov. 1, 2012

(54) TASTE MASKED PHARMACEUTICAL FORMULATIONS

(75) Inventors: Vinod Kumar Reddy Bondu, (US); Arun Arun Karunanidhi, (US); Subba Rao Malluru, (US); Ravi Kumar Namballa, (US); Srinivas Irukulla, (US); Harshal Prabhakar Bhagwatwar, (US)

(73) Assignee: DR. REDDY'S LABORATORIES LIMITED, Hyderabad (IN)

(21) Appl. No.: 13/435,458

(22) Filed: Mar. 30, 2012

Related U.S. Application Data
(60) Provisional application No. 61/486,345, filed on May 16, 2011.

(30) Foreign Application Priority Data
Apr. 1, 2011 (IN) 1142/CHE/2011

(51) Int. Cl. A61K 9/00; A61P 17/00; A61P 11/02; A61K 31/445; A61K 9/46

(52) U.S. Cl. 424/465; 424/400; 514/317

(57) ABSTRACT

The present invention relates to taste masked pharmaceutical formulations, processes for preparing the same and methods of using them. The present invention relates to taste masked effervescent formulations comprising fexofenadine or a pharmaceutically acceptable salt thereof, processes for preparing the same, and therapeutic uses and methods of treatment employing such formulations.

### INTRODUCTION

Aspects of the present application relate to taste masked pharmaceutical formulations comprising fexofenadine or pharmaceutically acceptable salts, esters, hydrates, solvates, derivatives, or single enantiomers thereof, and processes for preparing the same. In particular aspects, the present application relates to oro-dispersible effervescent tablets comprising fexofenadine hydrochloride, and processes for preparing the same. Aspects of the application further relate to therapeutic uses and methods of treatment employing such formulations comprising fexofenadine.

The drug compound having the adopted name “fexofenadine hydrochloride” has a chemical name: (±)-4-[1 hydroxy-4-[4-(hydroxydiphenylmethyl)-1-piperidinyl]-butyl]-α,α-dimethyl benzeneacetic acid hydrochloride; and is represented by structural Formula A.

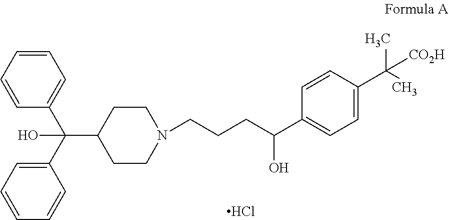

Fexofenadine hydrochloride is a white to off-white crystalline powder. It is freely soluble in methanol and ethanol, slightly soluble in chloroform and water, and insoluble in hexane.

Fexofenadine hydrochloride is a racemate and exists as a zwitterion in aqueous media at physiological pH.

Fexofenadine, a metabolite of terfenadine, is an antihistamine with selective peripheral H1-receptor antagonist activity. Fexofenadine is prescribed for treating seasonal allergic rhinitis and chronic idiopathic urticaria.

Fexofenadine is the active ingredient in Allegra® ODT tablets, marketed by SanofiAventis for oral administration and containing 30 mg of fexofenadine hydrochloride.

U.S. Pat. No. 4,254,129 discloses fexofenadine generically, its pharmaceutical composition, and a method of treating allergic reactions. U.S. Pat. No. 5,578,610 discloses piperidine derivatives.

U.S. Pat. No. 5,738,872 discloses a pharmaceutical composition prepared by a wet granulation process. U.S. Pat. No. 7,138,524 discloses anhydrous Form I and Forms II, III, and IV of fexofenadine hydrochloride, and a method of treating an allergic reaction in a patient by administering a solid unit dosage form comprising the Form I.

U.S. Pat. No. 5,503,846 discloses a stabilized effervescent dosage form comprising an acid sensitive pharmaceutically active agent, intimately mixed with a particulate effervescent pair of excipients, and the process of producing a particulate effervescent pair having an acid neutralization capacity less than about 5.

U.S. Pat. No 5,178,878 discloses a pharmaceutical dosage form comprising microparticles that are susceptible to rupturing upon chewing, or which are adapted to provide substantially immediate release of the pharmaceutical ingredient contained in the microparticles.

U.S. Pat. No. 6,723,348 discloses oro-dispersible tablets comprising coated granules of fexofenadine and the process for the preparation thereof.

U.S. Pat. No. 6,368,625 discloses a dosage form that rapidly disintegrates in the mouth and forms a viscous slurry, and also covers formulations of coated granules, coated particles, or microcapsules.

Several patents and other publications disclose different crystalline forms of fexofenadine, salts, and pharmaceutical compositions containing the same. Several other publications disclose methods of use of fexofenadine for various indications.

There remains a need for simple, taste masked, orally administrable pharmaceutical formulations comprising fexofenadine or pharmaceutically acceptable salts, esters, hydrates, solvates, derivatives, or single enantiomers thereof, for providing effective plasma concentrations of the active agent, and that are easy to manufacture.

### SUMMARY

Aspects of the present application relate to taste masked pharmaceutical formulations comprising fexofenadine or pharmaceutically acceptable salts, esters, hydrates, solvates, derivatives, or single enantiomers thereof, and processes for preparing the same.

In embodiments, the present application provides taste masked pharmaceutical formulations comprising fexofenadine hydrochloride, together with one or more pharmaceutical excipients.

Embodiments of the present application provide taste masked pharmaceutical formulations comprising crystalline fexofenadine hydrochloride Form X, described in U.S. Pat. No. 7,700,779, having mean particle sizes in the range of about 1 to about 50 μm.

Taste masked pharmaceutical formulations of the present application may be prepared in the form of chewable tablets, effervescent tablets, orally disintegrating, dispersible, dissolving, mucoadhesive, buccal, and sublingual tablets. In embodiments, taste masked fexofenadine hydrochloride formulations of the present application are in the form of orally disintegrating tablets.

In embodiments, the present application provides orally disintegrating tablet formulations comprising granules of fexofenadine, or a pharmaceutically acceptable salt thereof, and a mixture of excipients, wherein the granules comprise the active agent and one or more of excipients such as fillers, binders, lubricants, solvents, disintegrants, effervescent agents, diluents, sweeteners, flavors, and colorants.

In embodiments, the present application relates to pharmaceutical formulations comprising granules that contain fexofenadine hydrochloride, prepared using at least two binders in weight ratios ranging from about 1:10 to 10:1. In specific embodiments, two binders are ethyl cellulose and an aminoalkylmethacrylate copolymer.

In embodiments, taste masked pharmaceutical formulation of fexofenadine hydrochloride comprise insoluble fillers and soluble fillers. In embodiments, insoluble fillers are present intra-granularly and one or more soluble fillers are present extra-granularly. The insoluble fillers are present in concentrations about 0.1% to 20%, by weight of the total composition.

In embodiments, the application relates to taste masked formulation of fexofenadine hydrochloride, wherein combinations of one or more directly compressible and non-directly compressible fillers are used in weight ratios about 1:10 to 10:1

In embodiments, an effervescent pair is a mixture of an acidic agent and an alkaline agent, wherein weight ratios of acidic to alkaline components ranges from about 1:1 to about 1:5.

In embodiments, the present application relates to pharmaceutical formulations wherein one or more disintegrants are used in the range of about 1-20% by weight.

In embodiments, the present application relates to pharmaceutical formulations wherein one or more lubricants are present in the range of about 0.1% to 10% by weight.

In embodiments, taste masked formulations of fexofenadine hydrochloride contain one or more sugar and non-sugar sweeteners, combinations being in weight ratios about 1:10 to 10:1.

In embodiments, taste masked formulations of fexofenadine hydrochloride include one or more flavors.

An aspect of the present application provides stable pharmaceutical compositions of fexofenadine hydrochloride, wherein no polymorphic conversion of the drug is observed during manufacture and commercially relevant storage.

In embodiments, the present application provides taste masked pharmaceutical formulations of fexofenadine hydrochloride, which are substantially free of drug degradation impurities after commercially relevant storage periods.

In embodiments, the application relates to stable compositions and/or formulations wherein total drug-related impurities are present in amounts less than about 2%, or less than about 1%, of the label content of fexofenadine hydrochloride.

In embodiments, the present application includes taste masked solid oral dosage forms comprising fexofenadine hydrochloride, wherein the dosage forms release not less than 80% of the labeled amount of fexofenadine hydrochloride within about 30 minutes after immersion into an aqueous liquid.

In embodiments, drug-containing granules of the present application have particle sizes in the range of about 140-600 μm.

In embodiments, drug-containing granules of the present application have Carr index values in the range of about 1-50%.

In embodiments, the application provides methods of preparing taste masked formulation of fexofenadine hydrochloride.

In embodiments, taste masked orally disintegrating tablet formulation of the present application are useful in the treatment of symptoms associated with seasonal allergic rhinitis and chronic idiopathic urticaria, by administering oro-dispersible tablets to patients.

In embodiments, orally disintegrating tablets of the present application are bioequivalent to Allegra® ODT tablets.

### BRIEF DESCRIPTION OF THE DRAWINGS

FIG. 1 illustrates powder X-ray diffraction (PXRD) patterns described in Example 2A.

FIG. 2 illustrates PXRD patterns described in Example 2A.

FIG. 3 illustrates PXRD patterns described in Example 2B.

FIG. 4 illustrates PXRD patterns described in Example 2B.

FIG. 5 illustrates PXRD patterns described in Example 2B.

### DETAILED DESCRIPTION

As used herein, the term “fexofenadine” includes fexofenadine as a free base or in the form of a pharmaceutically acceptable salt, isomer, derivative, hydrate, solvate, or prodrug thereof.

In the present application, fexofenadine can be used in any crystalline form, amorphous form, or combinations thereof.

The term “excipient” or “pharmaceutically acceptable excipient” includes one or more of fillers, binders, diluents, lubricants, carriers, disintegrants, effervescent agents, sweeteners, flavors, colorants, etc.

The term “stability” as used in the description refers to polymorphic as well as chemical stability.

For the purposes of the present application an “orally disintegrating tablet” is a tablet capable of undergoing rapid disintegration in contact with saliva, without any chewing action, in less than 60 seconds, or in less than 40 seconds, forming a suspension that is easy to swallow.

Aspects of the present application provide taste masked pharmaceutical formulations comprising fexofenadine or a pharmaceutically acceptable salt thereof. In embodiments, fexofenadine is in the form of its hydrochloride salt. The formulations are particularly useful for elderly patients and children, who experience difficulties in deglutition of other solid dosage forms, even together with an intake of liquid.

Compositions of the application can be further processed into various pharmaceutical dosage forms as prepared, or can be combined with one or more pharmaceutically acceptable excipients. The different pharmaceutical dosage forms that comprise the pharmaceutical compositions of the present application include solid oral dosage forms such as, but not limited to, powders, granules, pellets, tablets, and capsules.

Taste masked pharmaceutical formulations may be incorporated into a number of dosage forms, including chewable, effervescent, orally disintegrating, dispersible, dissolving, mucoadhesive, buccal, and sublingual tablets.

The different physicochemical properties of the active ingredient, and as well as of excipients, are to be considered, as these properties affect the processing and formulation properties of the compound. Various important physicochemical properties include, but are not limited to, particle sizes, density (bulk density and tapped density), compressibility index, Hausner's ratio, angle of repose, etc.

Particle sizes of active pharmaceutical ingredient can affect a solid dosage form in numerous ways. For example, content uniformity (CU) of pharmaceutical dosage units can be affected by drug particle sizes and size distributions. This can be critical for low-dose drugs, and satisfactory dosage units of low doses cannot be manufactured from a drug that does not meet certain particle size and size distribution requirements. Also, particle sizes can play an important role in the dissolution of active ingredient from the final dosage form for certain drugs like fexofenadine, because of their poor solubility. Hence, these physicochemical properties not only affect the processes of preparing the pharmaceutical compositions, but also affect the performance of pharmaceutical products, both in vitro and in vivo.

The selection of appropriate particles sizes of fexofenadine, as well as of excipients, is within the scope of the application. The D10, D50, and D90 values are useful ways for indicating a particle size distribution. D90 is a size value, for which at least 90 volume percent of the particles have sizes smaller than the value. Likewise D10 refers to 10 volume percent of the particles having sizes smaller than the given value. D50 refers to at least 50 volume percent of the particles having sizes smaller than the given value, and D[4,3] refers to the mean particle size. Methods for determining D10, D50, D90, and D[4,3] include laser diffraction techniques, such as using equipment sold by Malvern Instruments Ltd., Malvern, Worcestershire, United Kingdom, or by Horiba.

In embodiments, the compositions of the present application comprise fexofenadine, or a pharmaceutically acceptable form of fexofenadine such as a salt, having particle size distributions such that: D90 is about 1 μm to about 100 μm, or about 1 μm to about 75 μm, or about 10 μm to about 50 μm; and D50 is from about 1 μm to about 50 μm, or about 1 μm to about 25 μm, or about 1 μm to about 15 μm.

Aspects of the present application relate to taste masked orally disintegrating effervescent tablets. In embodiments, the application accordingly provides orally disintegrating effervescent tablets comprising: at least one active principle present in non-effervescent granules, a mixture of excipients comprising at least one disintegrant, an effervescent pair, a diluent, a lubricant, and optionally any of sweeteners, flavors, and colorants.

Non-effervescent granules of the present application can be prepared using wet granulation processes, in fluidized bed processors. Non-effervescent granules comprise an active agent, a diluent, a binder, and an anti-adherent, wherein the active agent is mixed with the diluent and granulated using binder dispersion in a nonaqueous solvent.

Binder dispersions can be prepared by dispersing one or more binders and an anti-adherent in a nonaqueous solvent.

A non-aqueous solvent is an organic solvent that is substantially free of water. Suitable organic solvents include, but are not limited to, acetonitrile, chlorobenzene, chloroform, cyclohexane, 1,2-dichlorethane, dichloromethane, 1,2-dimethoxyethane, N,N,-dimethylacetamide, N,N-dimethylformamide, 1,4 dioxane, 2-ethoxyethanol, ethylene glycol, formamide, hexanes, 2-methoxyethanol, methyl butyl ketone, methylcyclohexane, N-methyl-pyrrolidone, nitromethane, pyridine, sulfolane, tetralin, toluene, 1,2-trichlorethane, xylene, acetic acid, acetone, anisole, butyl acetate, tert-butyl ethyl ether, cumene, dimethylsulfoxide, ethyl acetate, ethyl ether, ethyl formate, formic acid, heptanes, isobutyl acetate, isopropyl acetate, isopropyl alcohol, methyl acetate, methyl ethyl ketone, methyl isobutyl ketone, pentanel, propyl acetate, tetrahydrofuran, C₁-C₆ alcohols, and any mixtures of two or more thereof.

In embodiments, useful non-aqueous solvents of the present application include n-hexane and isopropanol.

Non-effervescent granules of the present application can be further processed with extragranular excipients and compressed to form tablets.

In embodiments, the extragranular excipients include at least one effervescent pair, which is a mixture comprising an acidic agent and an alkaline agent, one of which generates a gas by reacting with the other, when in contact with an aqueous fluid. In embodiments, the tablets undergo disintegration in the oral cavity in contact with saliva in less than 60 seconds, or in less than 40 seconds. In embodiments, an effervescent pair includes at least one alkaline member such as sodium bicarbonate, potassium bicarbonate, calcium carbonate, magnesium carbonate, sodium carbonate, and the like, and at least one acidic member such as citric acid, malic acid, fumaric acid, tartaric acid, phosphoric acid, alginic acid, and the like.

In embodiments, the combined amount of an effervescent pair in an orally disintegrating dosage form is from about 0.1 to about 20 percent, or about 2 to about 10 percent, by weight of the total disintegrating dosage form.

Various useful fillers or diluents according to the present application include, but are not limited to, starches, lactose, mannitol (e.g., Pearlitol® SD200), cellulose and its derivatives, confectioner's sugar and the like. Different grades of lactose include, but are not limited to, lactose monohydrate, lactose DT (direct tableting), lactose anhydrous, Flowlac™ (available from Meggle Products), Pharmatose™ (available from DMV), and others. Different starches include, but are not limited to, maize starch, potato starch, rice starch, wheat starch, pregelatinized starch (e.g., PCS PC10 from Signet Chemical Corporation) and starch 1500, starch 1500 LM grade (low moisture content grade) from Colorcon, fully pregelatinized starch (e.g., National 78-1551 from Essex Grain Products), and others. Different cellulose compounds that can be used include crystalline celluloses and powdered celluloses. Examples of crystalline cellulose products include, but are not limited to, Ceolus™ KG801, Avicel™ PH101, PH102, PH301, PH302 and PH-F20, PH-112, microcrystalline cellulose 114, and microcrystalline cellulose 112. Other useful diluents include, but are not limited to, carmellose, sugar alcohols such as sorbitol and xylitol, calcium carbonate, magnesium carbonate, dibasic calcium phosphate, and tribasic calcium phosphate.

Various useful binders according to the present application include, but are not limited to, hydroxypropyl celluloses, also called HPC (e.g., Klucel™ LF, Klucel™ EXF) and useful in various grades, hydroxypropyl methylcelluloses, also called hypromelloses or HPMC (e.g., Methocel™ products) and useful in various grades, polyvinylpyrrolidones or povidones (such as grades PVP-K25, PVP-K29, PVP-K30, and PVP-K90), copovidones (e.g., Plasdone™ S 630), powdered acacia, gelatin, guar gum, carbomers (e.g., Carbopol® products), methylcelluloses, ethylcelluloses, polymethacrylates, aminoalkylmethacrylate copolymers, starches, and mixtures thereof.

Various useful disintegrants include, but are not limited to, carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (Ac-di-sol™ from FMC-Asahi Chemical Industry Co., Ltd.), crospovidones, examples of commercially available crospovidone products including, but not limited to, crosslinked povidones, Kollidon® CL [manufactured by BASF (Germany)], Polyplasdone™ XL, XI-10, and INF-10 [manufactured by ISP Inc. (USA)], and low-substituted hydroxypropyl celluloses. Examples of low-substituted hydroxypropyl celluloses include, but are not limited to, low-substituted hydroxypropyl cellulose LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starches.

Lubricants can be added to granules both during the final mixing phase before compression and during granulation. Among the traditional solid lubricants, calcium, magnesium, and zinc salts of stearic acid, partially hydrogenated vegetable oils, polyethylene glycols, polyoxyethylene monostearate, talc, sodium benzoate, sodium laurylsulfate, magnesium oxide can be used. The amount of lubricant to be added typically varies from about 0.2% to 1% of the total composition.

Examples of useful sweeteners include aspartame, saccharin sodium, dipotassium glycyrrhizinate, stevia, thaumatin, acesulfame K, and sucralose.

A flavoring agent can be included during a final mixing of granules with other excipients or, making use of the fluidized bed granulation technique, it can be sprayed onto a granulate and then on the active ingredient, exploiting in some cases the capability of taste-masking of the flavor of the active ingredient. The amount of flavoring agent used can vary from about 0.5 to 3%, based on the composition total weight.

The flavoring agents that can be used include natural and artificial flavors. These flavors may be one or more of synthetic flavor oils and flavoring aromatics, and/or oils, oleo resins and extracts derived from plants, leaves, flowers, fruits, etc., and combinations thereof. Representative flavor oils include: spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavors such as vanilla, chocolate, coffee, cocoa and citrus oil, including lemon, orange, grape, lime and grapefruit and fruit essences including apple, pear, peach, strawberry, raspberry, cherry, plum, pineapple, apricot and so forth. These flavorings can be used individually or in admixture. Commonly used flavors also include mints such as peppermint, artificial vanilla, cinnamon derivatives, and various fruit flavors, whether employed individually or in admixture. Flavorings such as aldehydes and esters including cinnamyl acetate, cinnamaldehyde, citral, diethylacetal, dihydrocarvyl acetate, eugenyl formate, p-methylanisole, and so forth may also be used. Generally, any flavoring or food additive, such as those described in Chemicals Used in Food Processing, Publication 1274 of the National Academy of Sciences, pages 63-258, may be used. Further examples of aldehyde flavorings include, but are not limited to: acetaldehyde (apple); benzaldehyde (cherry, almond); cinnamic aldehyde (cinnamon); citrals, e.g., alpha-citral (lemon, lime); neral, beta-citral (lemon, lime); decanal (orange, lemon); ethyl vanillin (vanilla, cream); heliotropine, i.e., piperonal (vanilla, cream); vanillin (vanilla, cream); alpha-amyl cinnamaldehyde (spicy fruity flavors); butyraldehyde (butter, cheese); valeraldehyde (butter, cheese); citronellal (modifies, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e. trans-2 (berry fruits); tolyl aldehyde (cherry, almond); veratraldehyde (vanilla); 2,6-dimethyl-5-heptenal, i.e. melonal (melon); 2-6-dimethyloctanal (green fruit); and 2-dodecenal (citrus, mandarin); cherry; grape; mixtures thereof; and the like.

Pharmaceutical formulations of the present application can optionally contain coloring agents such as titanium dioxide, acceptable natural or synthetic coloring agents, etc., which can be added during the granulation phase or at any other point in a process.

Flowability of materials is measured and represented using the Carr Index. The Carr Index is the percentage ratio of the difference between tapped density and bulk density to tapped density, calculated as:

Carr Index=[(Tapped density-Bulk density)Tapped density]×100.

Carr Index values below about 15% represent materials with very good flow properties and values above about 40% represent materials with very poor flow properties. In embodiments, granules of fexofenadine compositions have a Carr Index which is substantially lower than the 40% described for products with poor flow properties. Values of Carr Index for fexofenadine HCl granules of the application are generally less than about 40%, or less than about 30%, or less than about 20%. This indicates superior handling capabilities during processing into pharmaceutical dosage forms.

The densities can be determined using standard test method 616 “Bulk Density and Tapped Density” from United States Pharmacopeia 32, United States Pharmacopeial Convention, Inc., Rockville, Md., 2009.

The dosage forms can be subjected to in-vitro dissolution testing, such as according to Test 711 “Dissolution” in United States Pharmacopeia 32, United States Pharmacopeial Convention, Inc., Rockville, Md., 2009 (“USP”), to determine the rate at which the active substance is released from the dosage forms, and content of active substance can be determined in dissolution media using techniques such as high performance liquid chromatography (HPLC).

An environment that a dosage form is likely to encounter when administered to a human (in vivo) can be correlated to in vitro dissolution studies conducted using various dissolution media, such as, but not limited to, simulated gastric fluid (SGF) with or without pepsin, simulated intestinal fluid (SIF) with or without pancreatin, 0.01 N hydrochloric acid (HCl), pH 1.2, 4.5, 5.5, 6.0, 6.8, 7.2, and 7.4 buffers, pH 2.1 SGF, pH 5.0 and 4.5 acetate buffers, pH 4.5 ammonium acetate buffer, pH 5.0 fed state simulated intestinal fluid (FeSSIF), pH 6.5 fasted state simulated intestinal fluid (FASSIF), pH 6.8 phosphate buffer, with or without sodium lauryl sulphate (SLS), pH 1.5 HCl buffer, and the like.

In embodiments, the present application comprises taste masked solid oral dosage forms comprising fexofenadine or pharmaceutically acceptable salts thereof, wherein the dosage forms release at least 25% of their contained active ingredient within about 10 minutes, and at least 40% of their contained active ingredient within about 30 minutes, after immersion in 500 mL of 0.001 N HCl (pH 3) dissolution medium using USP apparatus 2 (paddle) with 50 rpm stirring.

In embodiments, the present application comprises taste masked solid oral dosage forms comprising fexofenadine or pharmaceutically acceptable salts thereof, wherein the dosage forms release at least 25% of their contained active ingredient within about 10 minutes, and at least 40% of their contained active ingredient within about 30 minutes, after immersion in 500 mL of SGF without surfactant (pH 2.1) dissolution medium using USP apparatus 2 (paddle) at 50 rpm.

In embodiments, the present application comprises taste masked solid oral dosage forms comprising fexofenadine or pharmaceutically acceptable salts thereof, wherein said dosage forms release at least 25% of their contained active ingredient within about 10 minutes, and at least 40% of their contained active ingredient within about 30 minutes, after immersion in 500 mL of FaSSIF (pH 6.5) dissolution medium using USP apparatus 2 (paddle) at 50 rpm.

Various degradants and impurities that may be present in a fexofenadine containing formulation include the compounds having Formulas I-IV.

Benzene acetic acid, 4-[1-oxy-4-[4-(hydroxydiphenylmethyl)-1-piperidinyl]butyl]-α,α-dimethyl (Formula I).

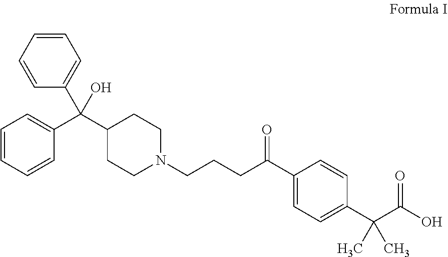

3-[1-Hydroxy-4-[4-(hydroxydiphenylmethyl)-1-piperidinyl]butyl]-α,α-dimethyl benzeneacetic acid hydrochloride (Formula II).

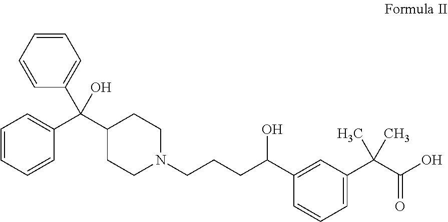

(±)-4-[1-Hydroxy-4-[4-(hydroxydiphenylmethyl)-1-piperidinyl]-butyl]isopropylbenzene (Formula III).

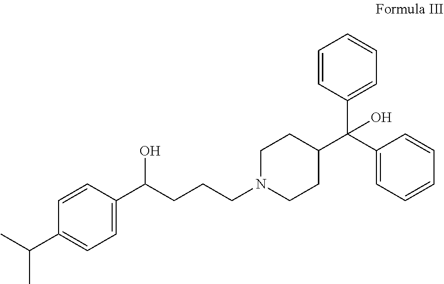

(RS)-2-[4-[1-Hydroxy-4-[4-(hydroxy-diphenyl-methyl)-1-piperidyl]butyl]phenyl]-2-methyl-propanoic acid N-oxide (Formula IV).

[symbol]

### Formula IV

In embodiments, the present application provides formulations of fexofenadine hydrochloride that are substantially free of individual degradation impurities after storage for commercially relevant times.

Pharmaceutical products are required to maintain stability after manufacturing for times such as 6 months, 1 year, 2 years, etc., when stored in their original packaging at normal ambient temperatures and humidity levels. It is customary in the industry to simulate this commercially relevant storage by storing at 25° C. and 60% RH, and the “accelerated stability” conditions of 40° C. and 75% RH, and measuring the physical properties as well as drug degradation product concentrations.

The term “substantially free” means presence of one or more degradation impurities in amounts less than about 5%, or about 4%, or about 3%, or about 2%, or about 1%, of the label content of fexofenadine hydrochloride.

In embodiments, the application relates to stable compositions and/or formulations wherein amounts of compound of Formula I are not more than about 0.3% of the label content of fexofenadine, after storage.

In embodiments, the application relates to stable compositions and/or formulations wherein amounts of the compound of Formula II are not more than about 0.1% of the label content of fexofenadine, after storage.

In embodiments, the application relates to stable compositions and/or formulations wherein amounts of the compound of Formula III are not more than about 0.15% of the label content of fexofenadine, after storage.

In embodiments, the application relates to stable compositions and/or formulations wherein amounts of the compound of Formula IV are not more than about 0.3% of the label content of fexofenadine, after storage.

In embodiments, the application relates to stable compositions and/or formulations wherein the total fexofenadine-related impurities are present in amounts not more than about 2%, or not more than about 1%, of the label content of fexofenadine hydrochloride.

Fexofenadine, fexofenadine hydrochloride, and impurities, such as those described above as I-IV, can be analyzed using ultra performance liquid chromatography (UPLC) with gradient elusion. Two mobile phases are used. Mobile phase A is a triethylamine buffer adjusted to pH 7.0 with phosphoric acid, and mobile phase B is water and acetonitrile, in the volume ratio of ratio of 100:900, respectively. The various chromatographic parameters are as follows:

Column: Acquity BEH C18, 100×2.1 mm, 1.7 μm, or equivalent.

Flow rate: 0.4 mL per minute.

UV detector wavelength: 220 nm.

Column temperature: 30° C.

Injection volume: 1.5 μL.

Run time: 40 minutes.

Sampling points: 20 points/second.

The elution gradient of the mobile phases is as follows:

| Minutes | Mobile Phase A (%) | Mobile Phase B (%) |
|---|---|---|
| 0 | 75 | 25 |
| 10 | 75 | 25 |
| 15 | 65 | 35 |
| 33 | 40 | 60 |
| 35 | 20 | 80 |
| 36 | 75 | 25 |
| 40 | 75 | 25 |

Typical relative retention times observed for impurities I-IV and fexofenadine hydrochloride are 1.41, 1.22, 3.21, 0.71, and 1, respectively.

Typical relative response factor values observed for impurities I-IV and fexofenadine hydrochloride are 0.84, 0.82, 0.96, 0.82, and 1, respectively.

Orally disintegrating tablets of the application can be rapidly disintegrated in the mouth, and have a high mechanical resistance and low friability. The term friability refers to an index which provides a measure of the ability of a tablet to withstand both shock and abrasion without crumbling, during handling in manufacturing, packaging, shipping, and consumer use. The orally disintegrating tablets of the present application generally have friability not greater than 1%.

In embodiments, formulations of the application are oro-dispersible tablets with a small thickness, thus increasing the tablet surface and consequently the speed of disintegration. In particular embodiments of the application, oro-dispersible tablets have thicknesses less than about 30% of the major diameter. This small thickness facilitates the disintegration of the tablets, as well as its palatability. Thus, in particular embodiments, the oro-dispersible tablets of the application disintegrate in less than 20 seconds, or in less than 15 seconds, or in less than 10 seconds.

Aspects of the present application provide processes for preparing orodispersible formulations comprising fexofenadine or a pharmaceutically acceptable salt thereof.

In embodiments, pharmaceutical formulations of the present application can be prepared using any of techniques including wet granulation, dry granulation, spray granulation, direct compression, and the like. An orally disintegrating tablet of the present application may include appropriate amounts of a variety of additives used for production of preparations, provided that they do not interfere with the effects of the present application.

Equipment suitable for processing the pharmaceutical compositions of the present application include any one or more of rapid mixer granulators, planetary mixers, mass mixers, ribbon mixers, fluid bed processors, mechanical sifters, blenders, roller compacters, extrusion-spheronizers, compression machines, capsule filling machines, rotating bowls or coating pans, tray dryers, fluid bed dryers, rotary cone vacuum dryers, and the like, multi-mills, fluid energy mills, ball mills, colloid mills, roller mills, hammer mills, and the like, equipped with a suitable screen.

In embodiments, non-effervescent granules of fexofenadine compositions are prepared by a process comprising:

1. Sifting fexofenadine HCl and microcrystalline cellulose through a sieve.

2. Sifting ethylcellulose, Eudragit® EPO and magnesium stearate through a sieve.

3. Dissolving Eudragit® EPO and ethylcellulose in isopropyl alcohol and dispersing magnesium stearate in the solution.

4. Granulating the material of step 1 with the binder dispersion of step 3, such as in a fluidized bed processor.

5. Drying the granulated material.

In embodiments, an effervescent pair is prepared by:

1. Sifting citric acid and sodium bicarbonate through a sieve and blending. 2. Mixing the blend with about approximately 1.5% w/w of water and drying the mixture.

3. Milling the mixture and passing through a sieve.

In embodiments, non-effervescent granules containing fexofenadine and an effervescent pair are mixed together. The blend is combined with extragranular material including any one or more of disintegrants, fillers, lubricants, flavors, and sweeteners. Finally, the blend is compressed into tablets.

In embodiments, the application includes the use of packaging materials such as containers and closures of high-density polyethylene (HDPE), low-density polyethylene (LDPE) and or polypropylene and/or glass, and blisters or strips composed of moisture resistant aluminum, high-density polypropylene, or polyvinyl chloride and/or polyvinylidene dichloride.

In embodiments, the application provides packages suitable for commercial sale, which provide stability for the contained products during storage, transportation, and use.

Certain specific aspects and embodiments of the disclosure will be further explained by the following examples, being provided only for purposes of illustration and not to be construed as limiting the scope of the disclosure.

### EXAMPLE 1

### Fexofenadine Orally Disintegrating Tablets

mg/Tablet

| | | |
|---|---|---|
| Ingredient | 1A | 1B |

Intragranular

| | | |
|---|---|---|
| Fexofenadine hydrochloride | 30 | 30 |
| Microcrystalline cellulose (Avicel ™ | 60 | 60 |
| PH101) | | |
| Ethylcellulose (7 cps) | — | 40 |
| Magnesium oxide | — | 50 |
| Mannitol (Pearlitol ® 25C) | 10 | — |
| Eudragit ® EPO | 30 | — |
| Isopropyl alcohol* | q.s. | q.s. |

Extragranular

| | | |
|---|---|---|
| Citric acid anhydrous | 15 | 15 |
| Sodium bicarbonate | 20 | 20 |
| Crospovidone (Kollidone ® CL) | 92 | 92 |
| Microcrystalline cellulose (Avicel PH112) | 19.5 | 19.5 |
| Mannitol (Pearlitol ® 25C) | 10 | 10 |
| Spray dried mannitol (Pearlitol ® SD 200) | 162.75 | 112.75 |
| Aspartame | 9.5 | 9.5 |
| Sucralose | 2.8 | 2.8 |
| Natural and artificial orange flavor | 3 | 3 |
| Artificial vanilla flavor | 2 | 2 |
| Magnesium stearate | 3.45 | 3.45 |

*Evaporates during processing.

Manufacturing Procedure:

Granulation for 1A:

1. Sift fexofenadine HCl through a #24 mesh sieve and Avicel™ PH101 through a #30 mesh sieve, and blend the materials.

2. Sift mannitol (Pearlitol® 25C) and Eudragit® EPO through a #30 mesh sieve, dissolve the Eudragit® EPO in isopropyl alcohol with stirring, and disperse Pearlitol® 25C in the solution.

3. Granulate the material of step 1 with the binder dispersion of step 2.

4. Dry the granulated material at 45° C. until the loss on drying (LOD) is less than 2% w/w, and sift the dried granules through a #30 mesh sieve.

5. Mill the granules retained on the sieve through a Quadro Comil fitted with a 1 mm screen and combine all of the granules passing through a #30 mesh sieve for further processing.

Granulation for 1B:

1. Separately sift fexofenadine HCl through a #24 mesh sieve and Avicel™ PH101, ethyl cellulose, and magnesium oxide through a #30 mesh sieve.

2. Mix fexofenadine HCl and Avicel™ PH101.

3. Dissolve ethylcellulose in isopropyl alcohol and disperse magnesium oxide in the solution.

4. Granulate the material of step 2 with the binder dispersion of step 3.

5. Dry the material of step 4 in the FBP at 45° C. until LOD is less than 2% w/w and sift the dried granules through a #30 mesh sieve.

6. Mill the granules retained on the sieve through a Comill fitted with a 1 mm screen and combine all of the granules passing through a #30 mesh sieve for further processing.

Formulation with Granules:

1. Sift citric acid and sodium bicarbonate through a #24 mesh sieve. Melt the citric acid at 80° C. and disperse sodium bicarbonate in the melt.

2. Cool the melt, mill through a Quadra Comil and dry the material.

3. Sift the material of step 3 through a #30 mesh sieve in an environment of less than 40% relative humidity (RH). Store in sealed containers until subsequent use.

4. Sift crospovidone, mannitol (Pearlitol® 25C and Pearlitol® SD 200), aspartame, sucralose, and microcrystalline cellulose 112, through a #30 mesh sieve, and blend with granules of either of 1A or 1B.

5. Sift natural and artificial orange flavor and artificial vanilla flavor through a #40 mesh sieve and sift magnesium stearate through a #60 mesh sieve, and blend the materials.

6. Blend materials of steps 4 and 5, and compress into tablets having hardness in the range of 1-5 kp.

### EXAMPLE 2

### Fexofenadine Orally Disintegrating Tablets

| | mg/Tablet | | |
|---|---|---|---|
| Ingredient | 2A | 2B | 2C |

Intragranular

| | | | |
|---|---|---|---|
| Fexofenadine hydrochloride (Form X) | 30 | 30 | 30 |
| Microcrystalline cellulose (Avicel ™ | 60 | 80 | 60 |
| PH101) | | | |
| Mannitol (Pearlitol ® 25C) | 5 | 20 | 5 |
| Eudragit ® EPO | 30 | 20 | 30 |
| Polyvinylpyrrolidone (Kollidon ® K30 or | — | 5 | 5 |
| Kollidon ® K90) | | | |
| Isopropyl alcohol* | q.s. | q.s. | q.s. |

Extragranular

| | | | |
|---|---|---|---|
| Citric acid anhydrous | 15 | 20 | 25 |
| Sodium bicarbonate | 20 | 35 | 30 |
| Crospovidone USP (Kollidon ® CL) | 92 | 70 | 94 |
| Microcrystalline cellulose (Avicel ™ | 19.5 | 48 | 19.5 |
| PH102 or Avicel ™ PH112) | | | |
| Mannitol 25C | 10 | 10 | 30 |
| Spray dried mannitol (Pearlitol ® SD | 167.75 | 100.4 | 121.85 |
| 200) | | | |
| Aspartame | 9.5 | 9.5 | 9.5 |
| Sucralose | 2.8 | 2 | 2.8 |
| Natural and artificial orange flavor | 3 | 3.5 | 3 |
| Artificial vanilla flavor | 2 | 2 | 2 |
| Magnesium stearate | 3.45 | 4.6 | 2.35 |

*Evaporates during processing.

Manufacturing Procedure for 2A:

1. Sift fexofenadine HCl through a #24 mesh sieve and sift Avicel™ PH101 through a #30 mesh sieve, and blend.

2. Sift mannitol (Pearlitol® 25C) and Eudragit® EPO through a #30 mesh sieve, then dissolve the Eudragit® EPO in isopropyl alcohol and disperse mannitol in the solution.

3. Granulate the material of step 1 with the binder dispersion of step 2.

4. Dry the granulated material at 45° C. until loss on drying (LOD) is less than 2% w/w, and sift the dried granules through a #30 mesh sieve.

5. Mill the granules retained on the sieve through a Quadra Comil fitted with a 1 mm screen and combine all of the granules passing through a #30 mesh sieve for further processing.

6. Sift citric acid and sodium bicarbonate through a #24 mesh sieve, mix, and maintain in a tray dryer at 75° C. for 12 hours.

7. Pass the dried material of step 6 through a Quadra Comill and sift through a #30 mesh sieve in an environment of less than 40% RH. Store the material in a sealed container until use.

8. Sift crospovidone, mannitol (Pearlitol® 25C and Pearlitol® SD 200), aspartame, sucralose, and microcrystalline cellulose 112 through a #30 mesh sieve.

9. Blend the materials of steps 5, 7, and 8.

10. Sift natural and artificial orange flavor and artificial vanilla flavor through a #40 mesh sieve. Sift magnesium stearate through a #60 mesh sieve.

11. Mix the materials of step 10 and blend with the materials of step 9.

12. Compress the blend from step 11 into tablets having hardness in the range of 1-5 kilopond (kp).

Manufacturing Procedure for 2B and 2C:

1. Sift fexofenadine HCl through a #24 mesh sieve and Avicel™ PH 101 through a #30 mesh sieve, and blend.

2. Sift mannitol (Pearlitol® 25C), povidone, and Eudragit® EPO through a #30 mesh sieve and blend.

3. Dissolve the povidone in isopropyl alcohol and granulate the blend of step 1 using the solution.

4. Dissolve Eudragit® EPO in isopropyl alcohol with stirring, disperse mannitol (Pearlitol® 25C) in the solution, and granulate the material of step 3 with the dispersion.

5. Dry the granules of step 4 at 45° C. until LOD is less than 2%, and sift the dried granules through a #30 mesh sieve.

6. Mill the granules retained on the #30 mesh sieve of step 5 through a Quadra Comil fitted with a 1 mm screen and pass the milled granules through a #30 mesh sieve. Combine all granules passing through a #30 mesh sieve.

7. Sift citric acid and sodium bicarbonate through a #24 mesh sieve and blend thoroughly. Keep the blend in a tray dryer maintained at 75° C., for 12 hours.

8. Mill the dried material of step 7 in a Quadra Comil and sift through a #30 mesh sieve, in an area having relative humidity less than about 40%. Store the material in a moisture impervious container.

9. Sift crospovidone, mannitol (Pearlitol® 25C and Pearlitol® SD 200), aspartame, sucralose, and microcrystalline cellulose 112 through a #30 mesh sieve.

10. Blend materials of steps 6, 8, and 9. 11. Sift natural and artificial orange flavor and artificial vanilla flavor through a #40 mesh sieve. Sift magnesium stearate through a #60 mesh sieve.

12. Blend materials of step 11.

13. Blend materials of steps 10 and 12.

14. Compress the blend from step 13 into tablets having hardness in the range of 1-5 kp.

In-vitro dissolution studies are performed using tablets of 2A and 2B under the following conditions, and the results are shown in Table 1.

Medium: 0.001N HCl.

Volume: 500 mL.

Apparatus: USP apparatus 2 (paddle).

Speed: 50 rpm.

Temperature: 37.0±0.5° C.

TABLE 1

| Cumulative % of Drug Dissolved | |
|---|---|
| Example 2A | Example 2B |

| Hours | Avg. | % RSD | Avg. | % RSD |
|---|---|---|---|---|
| 0 | 0 | 0 | 0 | 0 |
| 5 | 70 | 2.6 | 75 | 7 |
| 10 | 80 | 3.3 | 84 | 6.6 |
| 15 | 82 | 1.7 | 87 | 5.9 |
| 30 | 93 | 1.6 | 91 | 5.5 |
| 45 | 93 | 1.6 | 94 | 5.3 |
| 60 | 94 | 1.9 | — | |

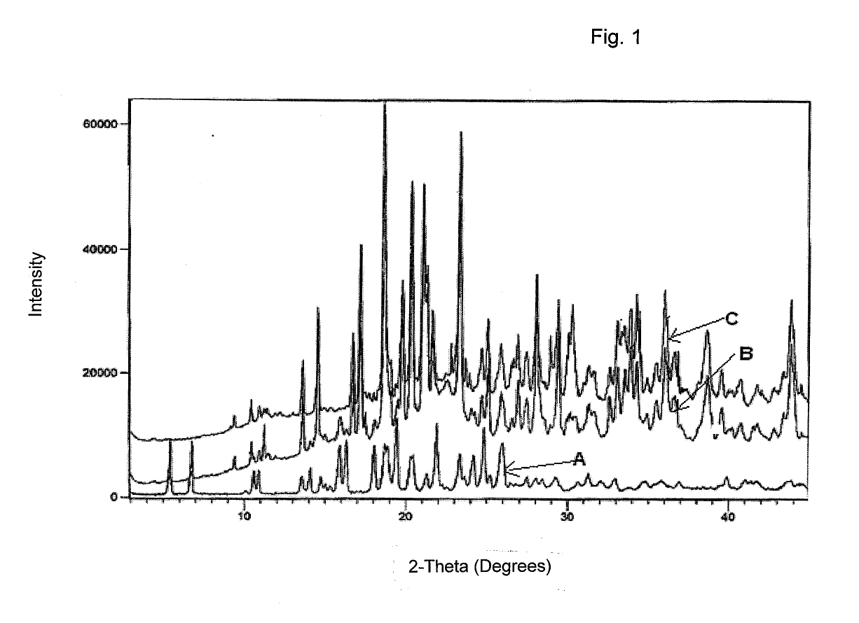

FIG. 1 illustrates PXRD patterns of: crystalline form X of fexofenadine hydrochloride (“A”); a placebo formulation prepared according to Example 2A, except for omitting the fexofenadine ingredient (“B”); and the tablet formulation of Example 2A (“C”). In the tablet PXRD data, no extra peaks are observed, indicating that polymorphic conversion of the drug does not occur during processing into a tablet formulation.

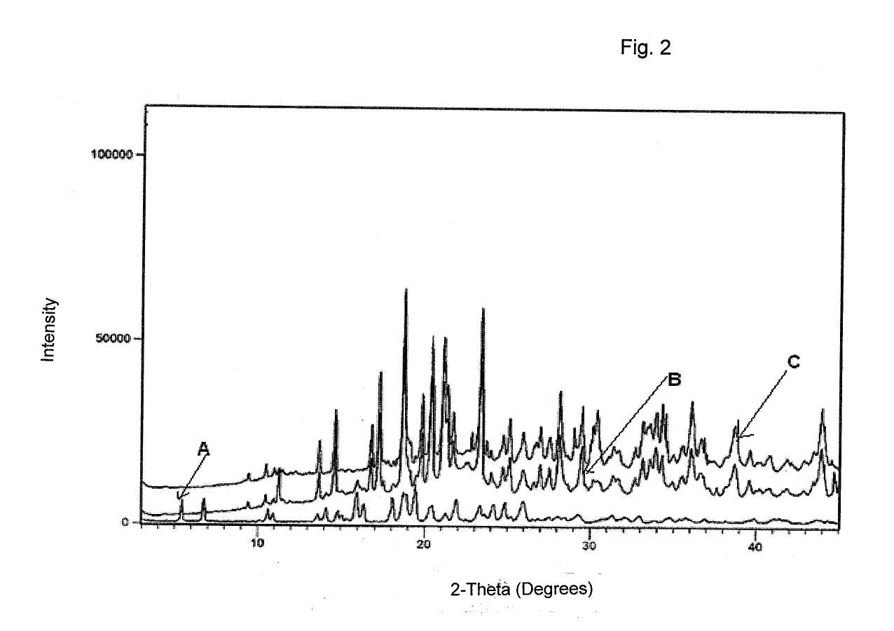

FIG. 2 illustrates PXRD patterns of: crystalline form X of fexofenadine hydrochloride (“A”); a placebo formulation prepared according to Example 2A, except for omitting the fexofenadine ingredient (“B”); and the tablet formulation of Example 2A (“C”) after stability testing storage at 40° C. and 75% relative humidity (RH) for 3 months. The PXRD data indicate that polymorphic conversion of the drug does not occur during storage.

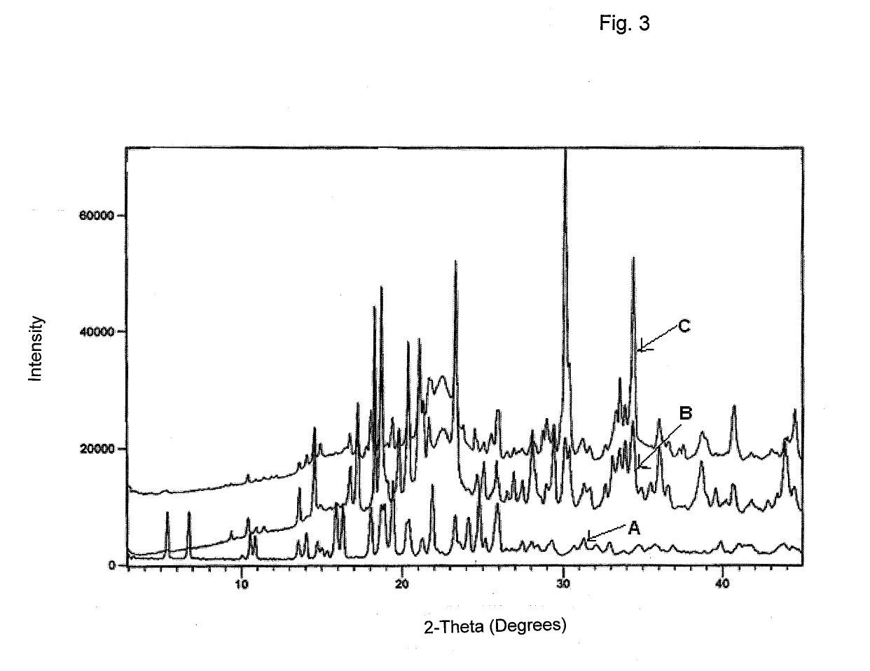

FIG. 3 illustrates PXRD patterns of: crystalline form X of fexofenadine hydrochloride (“A”); a placebo formulation prepared according to Example 2B, except for omitting the fexofenadine ingredient (“B”); and the tablet formulation of Example 2B (“C”). The PXRD data indicate that polymorphic conversion of the drug does not occur during processing into a tablet formulation.

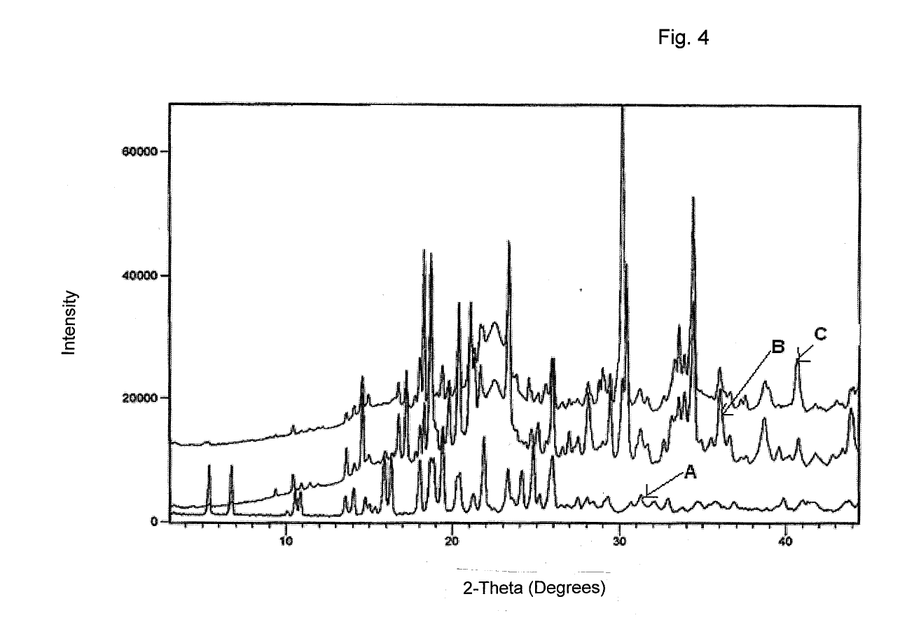

FIG. 4 illustrates PXRD patterns of: crystalline form X of fexofenadine hydrochloride (“A”); a placebo formulation prepared according to Example 2B, except for omitting the fexofenadine ingredient (“B”); and the tablet formulation of Example 2B (“C”) after stability testing storage at 40° C. and 75% RH for 1 month. The PXRD data indicate that polymorphic conversion of the drug does not occur during storage.

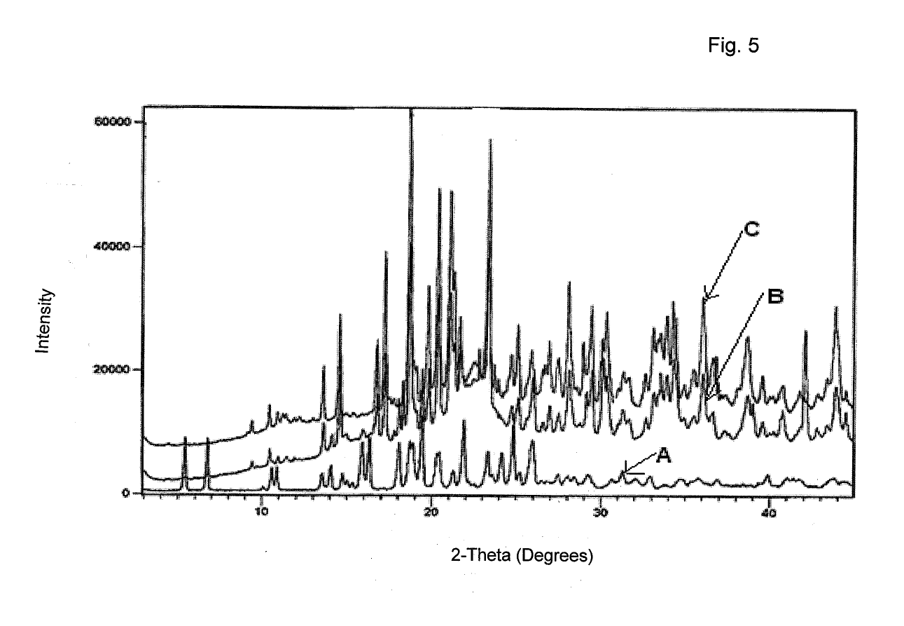

FIG. 5 illustrates PXRD patterns of: crystalline form X of fexofenadine hydrochloride (“A”); a placebo formulation prepared according to Example 2B, except for omitting the fexofenadine ingredient (“B”); and the tablet formulation of Example 2B (“C”) after stability testing storage at 40° C. and 75% RH for 3 months. The PXRD data indicate that polymorphic conversion of the drug does not occur during storage.

### EXAMPLE 3

### Fexofenadine Orally Disintegrating Tablets

| | |
|---|---|
| Ingredient | mg/Tablet |

Intragranular#

| | |
|---|---|
| Fexofenadine hydrochloride | 30 |
| Microcrystalline cellulose (Avicel ™ PH101) | 60 |
| Basic butylated methacrylate copolymer, Ph. Eur. | 35 |
| (Eudragit ® EPO) | |
| Isopropyl alcohol* | q.s. |

Extragranular

| | |
|---|---|
| Sodium bicarbonate | 5 |
| Crospovidone (Kollidon ® CL) | 93 |
| Microcrystalline cellulose (Avicel ™ PH112) | 25 |
| Mannitol (Pearlitol ® 25C) | 13 |
| Mannitol (Pearlitol ® 300DC) | 332.6 |
| Aspartame | 9.5 |
| Sucralose | 2.8 |
| Natural and artificial orange flavor | 5 |
| Artificial vanilla flavor | 4 |
| Prosweet ® N & A FL PWD‡ | 2 |
| Magnesium stearate | 3.1 |

#A 5% excess of these ingredients will be used, to compensate for processing losses.
‡Prosweet ® N & A FL PWD is a natural and artificial flavoring ingredient with dextrose as the carrier, from Virginia Dare, USA.
*Evaporates during processing.

Manufacturing Process:

1. Sift fexofenadine HCl and Avicel™ PH101 through a sieve and pass through a Quadra Comil fitted with a 2 mm screen. Pass the mixture again through the Comil.

2. Sift Eudragit EPO through a #24 mesh sieve and dissolve in isopropyl alcohol.

3. Granulate the material of step 1 with the binder solution of step 2.

4. Dry the granulated material at 45° C., until LOD is less than 2% w/w, and sift the dried granules through a #24 mesh sieve.

5. Sift sodium bicarbonate, crospovidone, mannitol (Pearlitol 25C and Pearlitol 300 DC), aspartame, sucralose, and microcrystalline cellulose 112 through a #30 mesh sieve.

6. Sift natural and artificial orange flavor, artificial vanilla flavor, and Prosweet through a #60 mesh sieve.

7. Blend materials of steps 4, 5, and 6.

8. Sift magnesium stearate through a #60 mesh sieve and blend with the material of step 7.

9. Compress the blend of step 8 into tablets having hardness in the range of 1-5 kp.

### EXAMPLE 4

### Fexofenadine Orally Disintegrating Tablets

| | mg/Tablet | | |
|---|---|---|---|
| Ingredient | 4A | 4B | 4C |

Intragranular#

| | | | |
|---|---|---|---|
| Fexofenadine hydrochloride | 30 | 30 | 30 |
| Microcrystalline cellulose (Avicel ™ PH101) | 30 | 30 | 60 |
| Isopropyl alcohol* | q.s. | q.s. | — |
| Microcrystalline cellulose (Avicel ™ PH102) | — | 90 | — |
| Mannitol (Pearlitol ® 200 SD) | 120 | — | — |
| Basic butylated methacrylate copolymer, | 40 | — | 35 |
| Ph. Eur. (Eudragit ® EPO) | | | |
| Ethylcellulose 7 cps | — | 25 | — |
| Magnesium oxide | — | 25 | — |
| Magnesium stearate | — | — | 2 |

Extragranular

| | | | |
|---|---|---|---|
| Sodium bicarbonate | 5 | 5 | 5 |
| Crospovidone (Kollidon ® CL) | 46.5 | 46.5 | 46.5 |
| Microcrystalline cellulose (Avicel ™ PH112) | — | — | 15 |
| Mannitol (Pearlitol ® 25C) | 50 | 50 | 50 |
| Mannitol (Pearlitol ® 300DC) | 269.8 | 289.8 | 349.8 |
| Aspartame | 9.5 | 9.5 | 9.5 |
| Natural and artificial orange flavor | 4.5 | 4.5 | 4.5 |
| Natural orange flavor | 4.5 | 4.5 | 4.5 |
| Artificial vanilla flavor | 4 | 4 | 4 |
| Magnesium stearate | 6.2 | 6.2 | 4.2 |

#A 5% excess of these ingredients will be used, to compensate for processing losses.
*Evaporates during processing.

Manufacturing Process for 4A and 4B:

1. Sift fexofenadine HCl and microcrystalline cellulose PH101 through a #16 mesh sieve.

2. Dissolve fexofenadine HCl in isopropyl alcohol and disperse Avicel™ PH101 in the solution.

3. Sift mannitol (Pearlitol® 200 SD) or microcrystalline cellulose PH102 through a #24 mesh sieve.

4. Adsorb the material of step 2 onto the material of step 3.

5. Dry the material of step 4 at 50° C., then pass the material through a Comil fitted with a 1.5 mm screen and sift through a #35 mesh sieve.

6. For 4A, Sift Eudragit EPO through a #24 mesh sieve and dissolve in isopropyl alcohol. For 4B, Dissolve ethylcellulose in isopropyl alcohol and disperse magnesium oxide in the solution.

7. Granulate the material of step 5 with the appropriate binder preparation of step 6.

8. Dry the granulated material at 45° C. until LOD is less than 2% w/w, then sift the dried granules through a #24 mesh sieve.

9. Sift sodium bicarbonate, crospovidone, mannitol (Pearlitol® 25 C) and aspartame through a #30 mesh sieve.

10. Blend materials of steps 8 and 9.

11. Sift mannitol (Pearlitol® 300 DC) through a #30 mesh sieve and blend with the material of step 10.

12. Sift magnesium stearate through a #60 mesh sieve and blend with the material of step 11.

13. Sift orange flavors and artificial vanilla flavor through a #40 mesh sieve and blend with the material of step 12.

14. Compress the blend from step 13 into tablets having hardness in the range of 1-5 kp.

15. Package the compressed tablets in aluminum foil blister packages or closed HDPE bottles.

Manufacturing Process for 4C: 1. Sift fexofenadine HCl and Avicel™ PH101 through a sieve and pass twice through a Quadra Comil fitted with a 2 mm screen.

2. Sift Eudragit® EPO and magnesium stearate through a #24 mesh sieve. Dissolve the Eudragit® EPO in isopropyl alcohol and disperse magnesium stearate in the solution.

3. Granulate the material of step 1 with the binder dispersion of step 2.

4. Dry the granulated material of step 3 at 45° C. until LOD is less than 2% w/w, and sift the dried granules through a #24 mesh sieve.

5. Blending, compression and packaging are performed similarly to the steps given for 4A and 4B.

### EXAMPLE 5

### Fexofenadine Orally Disintegrating Tablets

| | |
|---|---|
| Ingredient | mg/Tablet |

Intragranular#

| | |
|---|---|
| Fexofenadine hydrochloride | 30 |
| Microcrystalline cellulose (Avicel PH101) | 45 |
| Ethylcellulose (7 cps) | 25 |
| Magnesium oxide | 25 |
| Magnesium stearate | 15 |
| Isopropyl alcohol* | q.s. |

Extragranular

| | |
|---|---|
| Sodium bicarbonate | 10 |
| Citric acid anhydrous | 23 |
| Crospovidone (Kollidon ® CL) | 62 |
| Mannitol (Pearlitol ® 25C) | 50 |
| Mannitol (Pearlitol ® 300DC) | 313.9 |
| Aspartame | 9.5 |
| Natural and artificial orange flavor | 4.5 |
| Artificial vanilla flavor | 4 |
| Magnesium stearate | 3.1 |

#A 5% excess of these ingredients will be used, to compensate for processing losses.
*Evaporates during processing.

Manufacturing Process:

1. Sift fexofenadine HCl and microcrystalline cellulose through a #16 mesh sieve.

2. Dissolve ethyl cellulose in isopropyl alcohol and disperse magnesium oxide and magnesium stearate in the solution.

3. Granulate the material of step 1 with the binder dispersion of step 2.

4. Dry the granulated material at 45° C. until LOD is less than 2% w/w, and sift the dried granules through a #24 mesh sieve.

5. Sift sodium bicarbonate, citric acid, crospovidone, mannitol (Pearlitol® 25C), mannitol (Pearlitol® 300DC), and aspartame through a #24 mesh sieve.

6. Blend materials of steps 4 and 5.

7. Sift magnesium stearate through a #60 mesh sieve and blend with materials of step 6.

8. Sift orange flavor and artificial vanilla flavor through a #40 mesh sieve and blend with materials of step 7.

9. Blend materials of steps 7 and 8.

10. Compress the blend of step 9 into tablets having hardness in the range of 1-5 kp.

11. Package the tablets in aluminum blister packages or closed HDPE bottles.

### EXAMPLE 6

### Fexofenadine Orally Disintegrating Tablets

| | |
|---|---|
| Ingredient | mg/Tablet |

Intragranular#

| | |
|---|---|
| Fexofenadine hydrochloride | 30 |
| Microcrystalline cellulose (Avicel PH101) | 60 |
| Ethylcellulose (4 cps) | 35 |
| Eudragit ® EPO | 10 |
| Magnesium stearate | 5 |
| Isopropyl alcohol* | q.s. |

Extragranular

| | |
|---|---|
| Sodium bicarbonate | 10 |
| Citric acid anhydrous (granular) | 23 |
| Crospovidone (Kollidon ® CL) | 62 |
| Microcrystalline cellulose (Avicel ™ PH112) | 20 |
| Mannitol (Pearlitol ® 25C) | 50 |
| Mannitol (Pearlitol ® SD 200) | 292.35 |
| Aspartame | 9.5 |
| Natural and artificial orange flavor | 4.5 |
| Artificial vanilla flavor | 4 |
| Magnesium stearate | 4.65 |

#5% excess quantities of these ingredients will be used, to compensate for processing losses.
*Evaporates during processing.

Manufacturing Process:

1. Sift fexofenadine HCl and Avicel™ PH101 through a #16 mesh sieve and load into a FBP.

2. Sift ethylcellulose, Eudragit® EPO, and magnesium stearate through a #6 mesh sieve.

3. Dissolve Eudragit® EPO and ethylcellulose in isopropyl alcohol and disperse magnesium stearate in the solution. Pass the dispersion through a #40 mesh sieve and continuously stir throughout the granulation process.

4. Granulate the material of step 1 with the dispersion of step 3.

5. Dry the material of step 4 at 50° C. until LOD is less than 2% w/w, then sift the dried granules through a #24 mesh sieve. Use the material retained on the sieve for further processing.

6. Sift citric acid and sodium bicarbonate through a #20 mesh sieve and mix.

7. Load the material of step 6 into a steam jacketed kettle equipped with a propeller and add a small quantity of water (approximately 1.5% w/w) while mixing. Continue mixing for about 30 minutes.

8. Heat the material of step 7 at 80-90° C. for not less than 30 minutes, or until the LOD is less than 0.3% w/w.

9. Sift the dried material of step 8 through a #30 mesh sieve and pass the retained particles through a Quadra Comil fitted with a 1.5 mm screen, in an environment having RH less than 40%.

10. Pass the material of step 9 through a #30 mesh sieve in an environment of less than 40% RH. Store the material in a well-sealed container at less than 40% RH, for further use.

11. Sift crospovidone, mannitol (Pearlitol® 25C and Pearlitol® SD 200), aspartame, and microcrystalline cellulose 112 through a #20 mesh sieve.

12. Blend materials of steps 5, 10, and 11.

13. Sift magnesium stearate through a #20 mesh sieve and blend with the material of step 12.

14. Sift natural and artificial orange flavor and artificial cream flavor through a #20 mesh sieve and blend with the material of step 13.

15. Compress the blend from step 14 into tablets having hardness in the range of 2-5 kp.

16. Package the compressed tablets in aluminum blister packages or closed HDPE bottles.

In-vitro dissolution studies are performed using tablets of Example 6 under the following conditions, and the results are shown in Table 2.

Medium: 0.001N HCl.

Volume: 500 mL.

Apparatus: USP apparatus 2 (paddle).

Speed: 50 rpm.

Temperature: 37.0±0.5° C.

TABLE 2

Cumulative % of Drug Dissolved

| Hours | Avg. | % RSD |
|---|---|---|
| 0 | 0 | 0 |
| 5 | 45 | 4 |
| 10 | 73 | 2.5 |
| 15 | 85 | 2.2 |
| 30 | 96 | 1.6 |
| 45 | 98 | 1.9 |
| 60 | 99 | 1.7 |

Tablets of Example 6 are packaged and stored under the accelerated stability testing conditions of 40° C. and 75% RH for 3 months. The samples are analyzed for drug content and impurities of Formulas I-IV before, during, and after the storage, and results are shown in Table 3, where values are percentages of the label drug content.

TABLE 3

| | | Impurity | | | | | |
|---|---|---|---|---|---|---|---|
| | Drug | Formula | Formula | Formula | Formula | Highest | |
| Time | Assay | I | II | III | IV | Unidentified | Total |
| Initial | 99.9% | 0.049 | 0.048 | ND | ND | 0.015 | 0.112 |
| 1 month | 100.6% | 0.054 | 0.047 | ND | 0.040 | 0.017 | 0.158 |
| 2 months | 101.6% | 0.054 | 0.045 | ND | 0.051 | 0.016 | 0.166 |
| 3 months | 101.1% | 0.063 | 0.050 | ND | 0.058 | 0.017 | 0.188 |

ND = Not detected.

We claim:

1. A taste masked pharmaceutical formulation in a solid dosage form comprising an intragranular portion and an extragranular portion, wherein said intragranular portion comprises an active agent, at least two granulating aids in a ratio ranging from about 1:10 to about 10:1, and optionally one or more other pharmaceutically acceptable excipients; and said extragranular portion comprises an effervescent couple and one or more additional pharmaceutically acceptable excipients, wherein the effervescent couple consists essentially of an acidic substance and a basic substance in which the acidic substance is present in a molar excess amount.

2. The taste masked pharmaceutical formulation of claim 1, wherein intragranular portion contains at least one antiadherent.

3. The taste masked pharmaceutical formulation of claim 1, wherein the active agent is fexofenadine or a pharmaceutically acceptable salt thereof.

4. The taste masked pharmaceutical formulation of claim 1, wherein said intragranular portion and the extragranular portion are separated thereby substantially avoiding direct contact between the active agent and said effervescent couple.

5. The taste masked pharmaceutical formulation of claim 1, wherein said formulation disintegrates rapidly upon administration into the oral cavity.

6. The taste masked pharmaceutical formulation of claim 1, wherein one of said at least two granulating aids is a methacrylate polymer.

7. The taste masked pharmaceutical formulation of claim 1, wherein the solid dosage form is a tablet.

8. The taste masked pharmaceutical formulation of claim 1, wherein said formulation is in the form of uncoated tablet.

9. The taste masked pharmaceutical formulation of claim 1, wherein at least two granulating aids are present in a ratio ranging from about 1:5 to about 5:1.

10. The taste masked pharmaceutical formulation according to any of the preceding claims 1-9, wherein the granulating aid comprises at least one water insoluble polymer and at least one methacrylate polymer.

11. The taste masked pharmaceutical formulation of claim 10, wherein the water insoluble polymer is selected from a group comprising ethyl cellulose, hydroxyl propyl cellulose, methyl cellulose, and mixtures thereof.

12. The taste masked pharmaceutical formulation of claim 10, wherein the methacrylate polymer is a cationic copolymer based on dimethylaminoethyl methacrylate, butyl methacrylate, and methyl methacrylate.

13. The taste masked pharmaceutical formulation of claim 10, wherein the ratio of water insoluble polymer and methacrylate polymer is about 3.5:1.

14. The taste masked pharmaceutical formulation of claim 10, wherein methacrylate polymer is soluble at pH below 5.5.

15. The taste masked pharmaceutical formulation of claim 2, wherein the antiadherent is magnesium stearate.

16. The taste masked pharmaceutical formulation of claim 2, wherein the ratio of antiadherent to the granulating aids is about 1:10.

17. The taste masked pharmaceutical formulation of claim 1, wherein the intragranular portion comprises of at least one water soluble diluent or mixtures thereof.

18. The taste masked pharmaceutical formulation of claim 17, wherein the diluent is selected from sugar alcohols, cellulose compounds which include crystalline celluloses and powdered celluloses, lactose, starch, and mixtures thereof.

19. The taste masked pharmaceutical formulation of claim 17, wherein the water insoluble diluent in intragranular portion is present in concentrations of from about 20% to about 70%, by weight of the total weight of the intragranular portion.

20. The taste masked pharmaceutical formulation of claim 1, wherein the basic substance of the effervescent couple is selected from sodium bicarbonate, potassium bicarbonate, calcium carbonate, magnesium carbonate, sodium carbonate, and mixtures thereof.

21. The taste masked pharmaceutical formulation of claim 1, wherein the acidic substance of effervescent couple is selected from citric acid, malic acid, fumaric acid, tartaric acid, phosphoric acid, alginic acid, and mixtures thereof.

22. The taste masked pharmaceutical formulation of claim 1, wherein the basic substance is sodium bicarbonate and the acidic substance is citric acid.

23. The taste masked pharmaceutical formulation of claim 22, wherein the excess molar amount of citric acid is at least about 0.2×10⁻⁴ moles.

24. The taste masked pharmaceutical formulation of claim 1, wherein said extragranular portion comprises a disintegrant selected from crospovidone, microcrystalline cellulose, carboxymethylstarch sodium, croscarmellose sodium, sodium starch glycolate, colloidal silicon dioxide, starches, and mixtures thereof.

25. The taste masked pharmaceutical formulation of claim 1, wherein said extragranular portion comprises a diluent selected from various grades of starch, lactose, mannitol, cellulose compounds, sugar alcohols, calcium carbonate, magnesium carbonate, dibasic calcium phosphate, tribasic calcium phosphate, and mixtures thereof.

26. The taste masked pharmaceutical formulation of claim 25, wherein the extragranular diluent comprises a water soluble sugar alcohol which is mannitol.

27. The taste masked pharmaceutical formulation of claim 26, wherein the extragranular diluent comprises a mixture of two grades of mannitol one of which is a directly compressible grade and the other is a non-directly compressible grade, in a weight ratio of from about 1:10 to about 10:1.

28. The taste masked pharmaceutical formulation according to claims 1, wherein the extragranular portion comprises of one or more non-sugar sweeteners and/or one or more flavoring agents.

29. The taste masked pharmaceutical formulation of claim 1, wherein said extragranular portion comprises a lubricant selected from calcium, magnesium, and zinc salts of stearic acid; partially hydrogenated vegetable oils; polyethylene glycols;
polyoxyethylene monostearate; talc; and mixtures thereof.

30. The taste masked pharmaceutical formulation according to claim 1, wherein the drug-containing granules have an average particle size in the range of about 400-900 μm.

31. A taste masked pharmaceutical formulation in a solid dosage form comprising an intragranular portion and an extragranular portion wherein said intragranular portion comprises an active agent in an amount of about 10-30%, at least two granulating aids in a ratio ranging from about 1:10 to about 10:1 one of which is a methacrylate polymer in an amount of about 1-40%, and at least one antiadherent in an amount of about 2-20% by weight of the intragranular portion, optionally one or more other pharmaceutically acceptable excipients; an extragranular portion comprising an effervescent couple in an amount of about 2-30% by weight of the extragranular portion and one or more other pharmaceutically acceptable excipients, wherein the effervescent couple consists essentially of an acidic substance and a basic substance in which the acidic substance is present in a molar excess amount, such that an intimate contact between the active agent and the said effervescent couple is avoided; and wherein the said formulation rapidly disintegrates within about 1-2 minutes upon administration into the oral cavity.

32. The taste masked pharmaceutical formulation of claim 31, wherein the active agent is fexofenadine or a pharmaceutically acceptable salt thereof.

33. A process of preparation of taste masked pharmaceutical formulation in a solid dosage form comprising an intragranular portion and an extragranular portion wherein the process of making the intragranular portion comprises: a) mixing active agent and a diluent, b) granulating the blend of step a) with non aqueous dispersion comprising insoluble polymer and methacrylate polymer in a ratio ranging from 1:10 to 10:1 and an antiadherent, c) drying the granules; and process of preparing extragranular portion comprises: d) preparation of effervescent couple, e) sifting and blending other pharmaceutically acceptable excipients, and f) then mixing the granules of step c) with effervescent couple of step d) and the blend of step e); and finally compressing the blend of step f) to obtain a tablet dosage form.

34. The process of claim 33, wherein the active agent is fexofenadine or a pharmaceutically acceptable salt thereof.

35. A method of treatment of disease or disorder, wherein the method comprises administering to a subject in need thereof a taste masked pharmaceutical formulation according to claim 1.

36. A method of treatment of symptoms associated with seasonal allergic rhinitis and chronic idiopathic urticaria wherein the method comprises administering to a subject in need thereof a taste masked pharmaceutical formulation according to claim 1 comprising fexofenadine or a pharmaceutically acceptable salt thereof as an active agent.
